(19) United States
(12) Patent Application Publication
(10) Pub. No.: US 2025/0321553 A1
(43) Pub. Date: Oct. 16, 2025

(54) WIRELESS PHYSIOTHERAPY INSTRUMENT CONTROL METHOD, DEVICE AND MEDIUM

(71) Applicant: Chongqing Rob Linka Science and Technology Co, Ltd, Chongqing (CN)

(72) Inventors: Jianhua XU, Chongqing (CN); Wentao CHEN, Chongqing (CN)

(73) Assignee: Chongqing Rob Linka Science and Technology Co, Ltd, Chongqing (CN)

(21) Appl. No.: 19/177,296

(22) Filed: Apr. 11, 2025

(30) Foreign Application Priority Data
Apr. 15, 2024 (CN) 2024104486667

(51) Int. Cl. G05B 15/02

(52) U.S. Cl. CPC G05B 15/02

(57) ABSTRACT

The disclosure relates to a wireless physiotherapy instrument control method, and a device and a medium. The method comprises the following steps of: acquiring a control command sent by a physiotherapy instrument control end; determining a data transfer host corresponding to the control command from a plurality of physiotherapy instrument hosts; acquiring a plurality of control sub-commands comprised in the control command; aiming at each control sub-command in the plurality of control sub-commands, determining a target receiving host corresponding to each control sub-command from the plurality of physiotherapy instrument hosts; receiving the control command by using the data transfer host, and transferring each control sub-command in the control command to the corresponding target receiving host; and controlling each target receiving host to execute each corresponding control sub-command.

### CROSS-REFERENCE TO RELATED APPLICATIONS

The application claims priority to Chinese patent application No. 2024104486667, filed on Apr. 15, 2024, the entire contents of which are incorporated herein by reference.

### TECHNICAL FIELD

The present disclosure relates to the technical field of physiotherapy instrument control, in particular to a wireless physiotherapy instrument control method, a device and a medium.

### BACKGROUND

With the development of physiotherapy technology, people often use a wireless physiotherapy instrument to assist physiotherapy. A conventional wireless physiotherapy instrument generally outputs through a control end, receives an operation instruction through a physiotherapy instrument host, and then transmits a pulse current to a human body through a patch or other contact device connected with the physiotherapy instrument host to assist physiotherapy.

However, because the physiotherapy instrument control end is expensive, when there are a large number of hosts corresponding to the physiotherapy instrument control end, the physiotherapy instrument control end needs to send a large number of control commands, which consumes large electric quantity and internal storage, and accelerates damage to the physiotherapy instrument control end, leading to increased use cost of a user.

### SUMMARY

In order to solve a problem that, when there are a number of hosts corresponding to a physiotherapy instrument control end, the physiotherapy instrument control end needs to send a large number of control commands, which consumes large electric quantity and internal storage, and accelerates damage to the physiotherapy instrument control end, leading to increased use cost of a user, the present disclosure provides a wireless physiotherapy instrument control method, a device and a medium.

In a first aspect, in order to solve the above technical problem above, the present disclosure provides a wireless physiotherapy instrument control method, which includes the following steps of:
acquiring a control command sent by a physiotherapy instrument control end;
determining a data transfer host corresponding to the control command from a plurality of physiotherapy instrument hosts;
acquiring a plurality of control sub-commands included in the control command;
aiming at each control sub-command in the plurality of control sub-commands, determining a target receiving host corresponding to each control sub-command from the plurality of physiotherapy instrument hosts;
receiving the control command by using the data transfer host, and transferring each control sub-command in the control command to the corresponding target receiving host; and
controlling each target receiving host to execute each corresponding control sub-command.

In a second aspect, the present disclosure further provides a computing device, which includes a memory, a processor, and a program stored on the memory and operated on the processor, wherein the processor executes the program to implement the steps of the wireless physiotherapy instrument control method above.

In a third aspect, the present disclosure further provides a computer-readable storage medium having an instruction stored therein, which when running on a terminal device, causes the terminal device to perform the steps of the wireless physiotherapy instrument control method above.

The present disclosure has the beneficial effects that: the data transfer host is determined through the control command sent by the physiotherapy instrument control end, and the target receiving host corresponding to each control sub-command in the control command is determined. Subsequently, the control command is received by the data transfer host, and each control sub-command in the control command is sent to the corresponding target receiving host by the data transfer host. In this way, the physiotherapy instrument control end only needs to send one control command to one physiotherapy instrument host each time, so that the plurality of physiotherapy instrument hosts may be controlled to execute corresponding control sub-commands. This way of sending the control sub-commands to the plurality of target receiving hosts through the data transfer host reduces a use frequency of the physiotherapy instrument control end, so that less electric quantity and internal storage are consumed, and damage to the physiotherapy instrument control end is slowed down, thereby reducing use cost of a user.

### BRIEF DESCRIPTION OF DRAWINGS

FIG. 1 is a flow chart of a wireless physiotherapy instrument control method according to the present disclosure;

FIG. 2 is another flow chart of the wireless physiotherapy instrument control method according to the present disclosure;

FIG. 3 is another flow chart of the wireless physiotherapy instrument control method according to the present disclosure;

FIG. 4 is another flow chart of the wireless physiotherapy instrument control method according to the present disclosure;

FIG. 5 is a schematic structural diagram of a wireless physiotherapy instrument control system according to the present disclosure; and

FIG. 6 shows a network structure of the wireless physiotherapy instrument control system according to the present disclosure.

### DETAILED DESCRIPTION OF THE EMBODIMENTS

The following embodiments are further explanations and supplements to the present disclosure, and do not constitute any restrictions on the present disclosure.

A wireless physiotherapy instrument control method and system, a device and a medium according to embodiments of the present disclosure are described with reference to the drawings.

The wireless physiotherapy instrument control method according to the embodiments of the present disclosure is applied to a terminal device. A controller is taken as an execution subject in a solution of the present disclosure to describe the solution of the present disclosure, and the controller is used to execute steps of the wireless physiotherapy instrument control method.

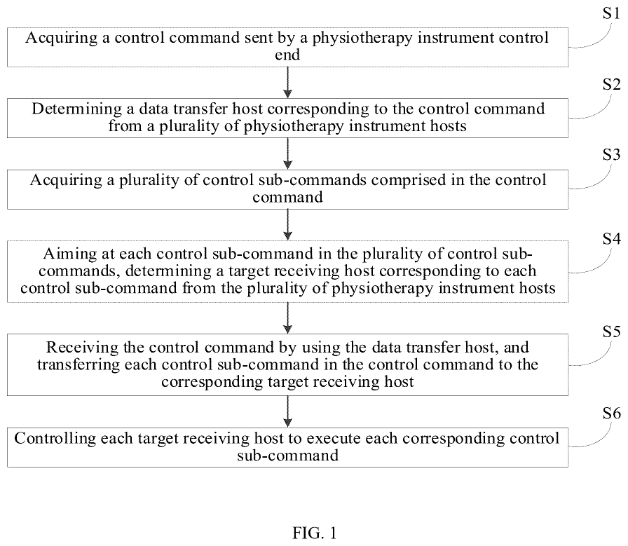

As shown in FIG. 1, the present disclosure provides a wireless physiotherapy instrument control method, which includes the following steps:
step S1: acquiring a control command sent by a physiotherapy instrument control end;
step S2: determining a data transfer host corresponding to the control command from a plurality of physiotherapy instrument hosts;
step S3: acquiring a plurality of control sub-commands included in the control command;
step S4: aiming at each control sub-command in the plurality of control sub-commands, determining a target receiving host corresponding to each control sub-command from the plurality of physiotherapy instrument hosts;
step S5: receiving the control command by using the data transfer host, and transferring each control sub-command in the control command to the corresponding target receiving host; and
step S6: controlling each target receiving host to execute each corresponding control sub-command.

According to the wireless physiotherapy instrument control method provided by the present disclosure, the data transfer host is determined through the control command sent by the physiotherapy instrument control end, and the target receiving host corresponding to each control sub-command in the control command is determined. Subsequently, the control command is received by the data transfer host, and each control sub-command in the control command is sent to the corresponding target receiving host by the data transfer host. In this way, the physiotherapy instrument control end only needs to send one control command to one physiotherapy instrument host each time, so that the plurality of physiotherapy instrument hosts may be controlled to execute corresponding control sub-commands. This way of sending the control sub-commands to the plurality of target receiving hosts through the data transfer host reduces a use frequency of the physiotherapy instrument control end, so that less electric quantity and internal storage are consumed, and damage to the physiotherapy instrument control end is slowed down, thereby reducing use cost of a user.

In some embodiments, two or more physiotherapy instrument hosts are provided, and one data transfer host is provided, which are determined according to an actual connection situation, and the data transfer host connected each time is not necessarily the same one. At least one or more target receiving hosts may be provided. The data transfer host may also be taken as one target receiving host to execute a corresponding control sub-command.

Optionally, the determining the data transfer host corresponding to the control command from the plurality of physiotherapy instrument hosts, includes:
acquiring transfer end information included in the control command; and
determining a first target host matched with the transfer end information in the plurality of physiotherapy instrument hosts, and taking the first target host as the data transfer host.

In this embodiment, the transfer end information in the control command is consistent with information of the data transfer host to be determined, so the first target host matched with the transfer end information can be determined by only matching the plurality of physiotherapy instrument hosts, and the first target host is taken as the data transfer host. Compared with the way of sequentially inquiring whether each physiotherapy instrument host is the data transfer host each time, the method in this embodiment can reduce time used and internal storage occupied for multiple data accesses by multiple data accesses, thereby improving the efficiency of determining the data transfer host.

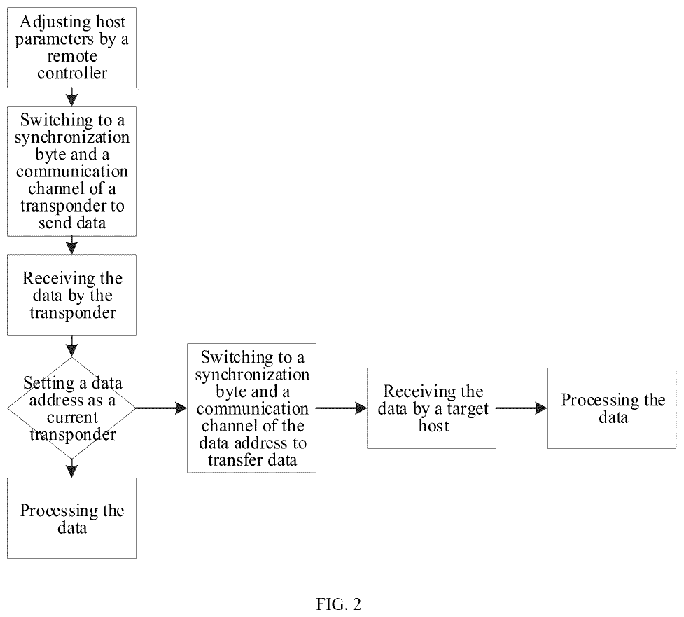

FIG. 2 is another flow chart of the wireless physiotherapy instrument control method according to the present disclosure. As shown in FIG. 2, a remote controller (the physiotherapy instrument control end) adjusts host parameters, and switches to a synchronization byte and a communication channel of a transponder (the data transfer host) to send data (the control command). After the physiotherapy instrument host (the transponder) receives the data, a data address of the physiotherapy instrument host receiving the data is taken as a current transponder (data transfer). The transponder carries out data processing on the received data, and meanwhile, the transponder switches the synchronization byte and the communication channel to a synchronization byte and a communication channel of the target receiving host, so as to transfer a part of the received data to the target receiving host. After the target receiving host receives the data, data processing is carried out on the received data, so as to realize the effect that the remote controller (the physiotherapy instrument control end) can control the plurality of physiotherapy instrument hosts by controlling only one transponder, thereby reducing the use frequency of the remote controller (the physiotherapy instrument control end) and prolonging the service life of the remote controller (the physiotherapy instrument control end).

Optionally, the transfer end information includes transfer end synchronization byte information and transfer end channel information.

The determining the first target host matched with the transfer end information in the plurality of physiotherapy instrument hosts, and taking the first target host as the data transfer host, includes:
acquiring host information of each physiotherapy instrument host in the plurality of physiotherapy instrument hosts, wherein the host information includes synchronization byte information and channel information;
taking host information which is the same as the transfer end information in multiple pieces of host information as first target host information; and
taking a physiotherapy instrument host corresponding to the first target host information as the first target host, and taking the first target host as the data transfer host.

In this embodiment, the synchronization byte and the channel information of each physiotherapy instrument host are different, and during data transmission, a data receiving end and a data transmitting end need to use the same communication channel to carry out data communication. Therefore, according to the transfer end synchronization byte information and the transfer end channel information in the transfer end information, and the synchronization byte information and the channel information of each physiotherapy instrument host, the data transfer host capable of carrying out data communication with the physiotherapy instrument control end can be directly matched, which is convenient for the physiotherapy instrument control end to transfer the corresponding control sub-commands to various target receiving hosts through the data transfer host, so as to achieve the purpose that the physiotherapy instrument control end controls all target receiving hosts by controlling one physiotherapy instrument host, thereby reducing the use frequency of the physiotherapy instrument control end and prolonging the service life.

In some embodiments, the synchronization byte is similar to a physical identification address of an MAC address, and when communication channels of the data sending end and the data receiving end are the same, while synchronization bytes of the data sending end and the data receiving end are different, the data sending end and the data receiving end cannot send and receive data mutually. The channel refers to the communication channel, and the remote controller and the host both save all synchronization byte and channel lists.

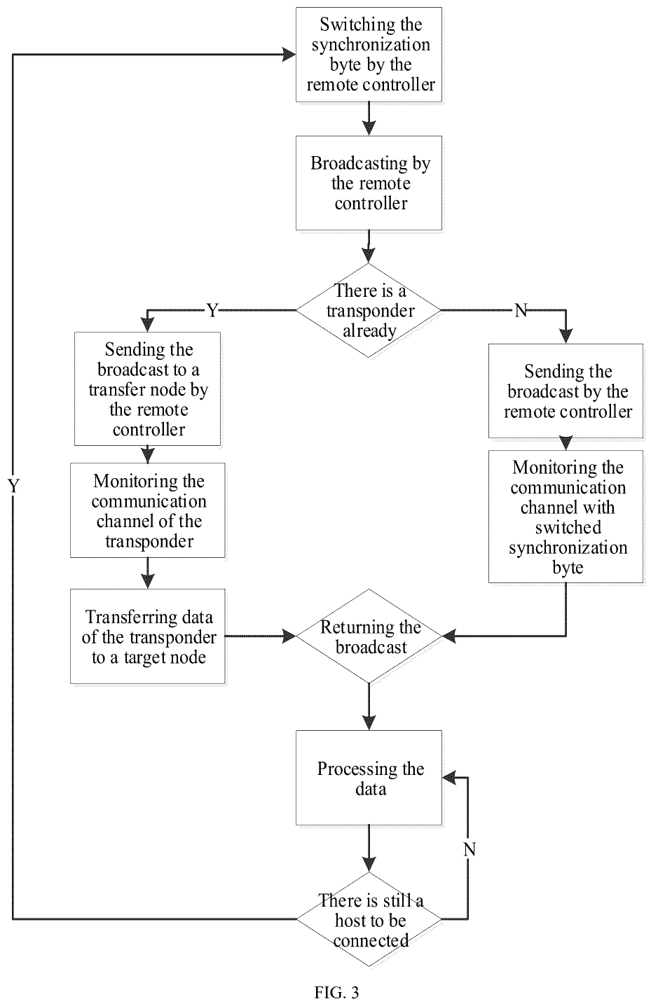

FIG. 3 is another flow chart of the wireless physiotherapy instrument control method according to the present disclosure. As shown in FIG. 3, the remote controller (the physiotherapy instrument control end) selects and switches to the synchronization byte of each host to be connected (the target receiving host), so as to form the control sub-command of each host to be connected, and the control command containing various control sub-commands is broadcast (the control command is sent). If there is no transponder, the remote controller sends a broadcast (sends the control command) to determine a transponder, and then monitor the transfer end information and switches to the synchronization byte and the communication channel of the determined transponder. If there is a transponder, the control command is sent to the transponder (the data transfer host), the communication channel of the transponder is monitored at the same time, and then the transponder transfers various control sub-commands in the control command to the corresponding host to be connected (the target receiving host). Various hosts to be connected generate execution data after executing the corresponding control sub-commands, and the execution data are sent back to the transponder by broadcasting. The transponder carries out data processing on the execution data sent back. In this case, there is still a physiotherapy instrument host to be connected, and the physiotherapy instrument control end continues to broadcast through the data transfer host (the data transfer host transfers the control sub-commands in the control command) to other physiotherapy instrument hosts with different channels and different synchronization bytes. After receiving the broadcast data (the control sub-commands), an unconnected physiotherapy instrument host executes according to the broadcast data to obtain the execution data, and then the data transfer host feeds back the execution data to the physiotherapy instrument control end for data interaction to establish connection.

Optionally, the aiming at each control sub-command in the plurality of control sub-commands, determining the target receiving host corresponding to each control sub-command from the plurality of physiotherapy instrument hosts, includes:
aiming at each control sub-command in the plurality of control sub-commands, acquiring receiving end information included in each control sub-command; and
determining a second target host matched with the receiving end information in the plurality of physiotherapy instrument hosts, and taking the second target host as the target receiving host corresponding to each control sub-command.

In this embodiment, the receiving end information in each control sub-command is consistent with information of the target receiving host to be determined, then only the plurality of physiotherapy instrument hosts need to be matched to determine the second target host matched with each piece of receiving end information, and the second target host is taken as the target receiving host corresponding to each control sub-command. Compared with the way of inquiring whether each physiotherapy instrument host is the target receiving host for each control sub-command, the method in this embodiment can reduce the time used and the internal storage occupied for multiple data accesses by multiple data accesses, thereby improving the efficiency of determining the target receiving host corresponding to each control sub-command.

Optionally, the receiving end information includes receiving end synchronization byte information and receiving end channel information.

The determining the second target host matched with the receiving end information in the plurality of physiotherapy instrument hosts, and taking the second target host as the target receiving host corresponding to each control sub-command, includes:
acquiring host information of each physiotherapy instrument host in the plurality of physiotherapy instrument hosts, wherein the host information includes synchronization byte information and channel information;
taking host information which is the same as the receiving end information in multiple pieces of host information as second target host information; and
taking a physiotherapy instrument host corresponding to the second target host information as the second target host, and taking the second target host as the target receiving host corresponding to each control sub-command.

In this embodiment, the synchronization byte and the channel information of each physiotherapy instrument host are different, and during data transmission, a data receiving end and a data transmitting end need to use the same communication channel to carry out data communication. Therefore, according to the receiving end synchronization byte information and the receiving end channel information in the receiving end information, and the synchronization byte information and the channel information of each physiotherapy instrument host, the target receiving host capable of receiving each control sub-command can be directly matched, so as to realize the communication of each control sub-command in unique route between the corresponding target receiving host and the data transfer host, which is convenient to realize accurate transmission and safe transmission of each control sub-command subsequently, thereby improving the communication efficiency of the data transfer host with various target receiving hosts.

Optionally, the method further includes:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands included in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

In this embodiment, when the data transfer host is in an abnormal state, the data transfer host cannot receive the control command sent by the physiotherapy instrument control end, and/or transfer various control sub-commands in the control command, so that it is necessary to re-acquire the new control command sent by the physiotherapy instrument control end, and the new data transfer host corresponding to the new control command is determined from the plurality of physiotherapy instrument hosts until the new data transfer host is not in the abnormal state. In this way, when the data transfer host is abnormal in communication, the data transfer host is replaced by the new data transfer host to receive the control command sent by the physiotherapy instrument control end and transfer various control sub-commands in the control command, so that when the physiotherapy instrument control end cannot control various target receiving hosts due to the abnormal communication of the original data transfer host, the data communication during controlling over various target receiving hosts by the physiotherapy instrument control end can be restored in time, so as to realize the timeliness and correct sending and reception of data transmission in a network.

Optionally, the when the data transfer host is in the abnormal state, acquiring the new control command sent by the physiotherapy instrument control end, includes:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

In this embodiment, when the data transfer host can receive the control command sent by the physiotherapy instrument control end, and transfer various control sub-commands in the control command, it is indicated that the data transfer host can send and receive data. At this time, after receiving the inquiry instruction sent by the physiotherapy instrument control end, the data transfer host will send the response information to the physiotherapy instrument control end. When the data transfer host is in the abnormal state, the data transfer host cannot receive the control command sent by the physiotherapy instrument control end, and/or transfer various control sub-commands in the control command, and it is indicated that a date sending and receiving function of the data transfer host is abnormal. At this time, after receiving the inquiry instruction sent by the physiotherapy instrument control end, the data transfer host will not send the response information to the physiotherapy instrument control end. In some cases, when there is network delay, instantaneous failure of the data transfer host and human interference in communication of the data transfer host, the data transfer host cannot send the response information to the physiotherapy instrument control end in time. Therefore, when the physiotherapy instrument control end does not receive the response information fed back by the data transfer host based on the inquiry instruction, the physiotherapy instrument control end is controlled to poll and send the inquiry instruction according to the preset period, and the number of sending times of the inquiry instruction polled and sent is recorded. If the number of sending times is less than the preset threshold, it is indicated that the data transfer host can realize the data sending and receiving function. If the number of sending times reaches the preset threshold, it is indicated that the sending and receiving function of the data transfer host is abnormal. At this time, the new control command sent by the physiotherapy instrument control end is acquired until the newly determined data transfer host realize the data sending and receiving function, so that when the data transfer host is abnormal, the data transfer host can be replaced in time, and the data communication of the control command sent by the physiotherapy instrument control end can be restored in time, so as to timely control various target receiving hosts to execute the corresponding target sub-commands.

In some embodiments, the inquiry instruction is a sub-command in the control command sent by the physiotherapy instrument host, or a command to inquire whether the data transfer host can communicate with the physiotherapy instrument host and various target receiving hosts. The number of sending times includes the first time that the physiotherapy instrument control end sends the inquiry instruction to the data transfer host, and each time that the physiotherapy instrument control end is controlled to poll and send the inquiry instruction according to the preset period.

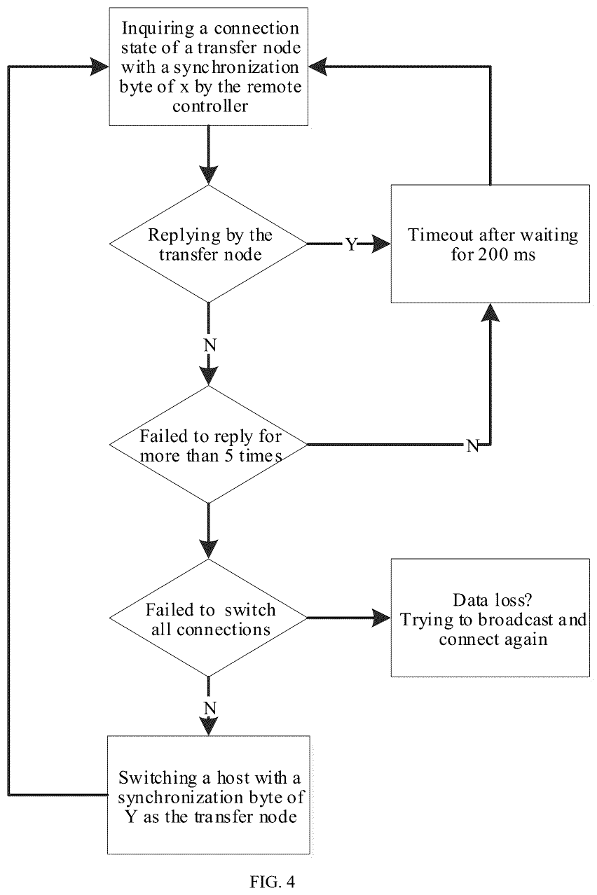

FIG. 4 is another flow chart of the wireless physiotherapy instrument control method according to the present disclosure. As shown in FIG. 4, the remote controller (the physiotherapy instrument control end) sends inquiry information to inquire a connection station of a transfer node (the data transfer host) with a synchronization byte same as X in the transfer end information, and this connection state represents a normal or abnormal data sending and receiving state. The remote controller waits for 200 ms. If the transfer node (the data transfer host) replies to the remote controller with the response information, the operation returns to the remote controller, and it is determined that the transfer node represents the normal data sending and receiving state. The remote controller sends the inquiry information once and waits for 200 ms, and the number of sending times of the inquiry information sent by the remote controller is recorded. If the transfer node fails to reply to the remote controller with the response information, the remote controller sends the inquiry information again and waits for 200 ms again, and the number of sending times of the inquiry information sent by the remote controller is recorded until the transfer node replies to the remote controller with the response information, and it is determined that the transfer node represents the normal data sending and receiving state. If the number of sending times reaches 5, that is, the transfer node fails to reply for more than 5 times, other physiotherapy instrument host is matched, and a new transfer node is connected. If the transfer connections of all the physiotherapy instrument hosts are failed to switch, the transfer node cannot be determined, which will cause data loss, and then the remote controller broadcasts again and sends the control command to connect the transfer node. If the transfer connections of the physiotherapy instrument hosts are not failed switch completely, the synchronization byte in the transfer end information is switched to Y which is the same as the synchronization byte of another physiotherapy instrument host, and the host with the synchronization byte of Y is taken as the transfer node. That is, the remote controller polls a state of the transfer host (once every 200 ms), and the host is switched to other connected host when no response is made for more than 5 times. After the transfer node is switched, the transfer node polls and informs that other host transfer nodes in network nodes have been switched until all nodes have been confirmed.

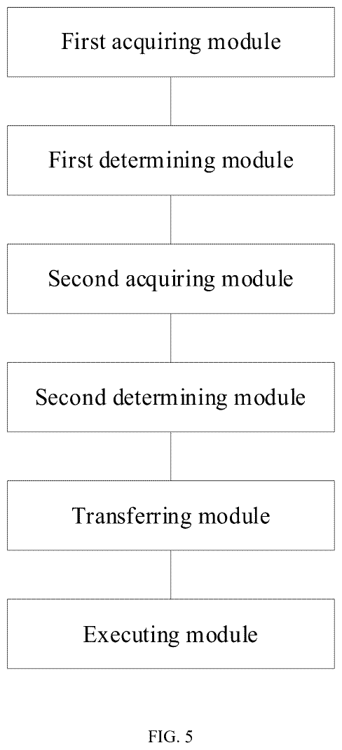

As shown in FIG. 5, the present disclosure provides a wireless physiotherapy instrument control system, which includes:
a first acquiring module, configured for acquiring a control command sent by a physiotherapy instrument control end;
a first determining module, configured for determining a data transfer host corresponding to the control command from a plurality of physiotherapy instrument hosts;
a second acquiring module, configured for acquiring a plurality of control sub-commands included in the control command;
a second determining module, configured for, aiming at each control sub-command in the plurality of control sub-commands, determining a target receiving host corresponding to each control sub-command from the plurality of physiotherapy instrument hosts;
a transferring module, configured for receiving the control command by using the data transfer host, and transferring each control sub-command in the control command to the corresponding target receiving host; and
an executing module, configured for controlling each target receiving host to execute each corresponding control sub-command.

Through the method and system provided by the present disclosure, the physiotherapy instrument control end is connected with one of the plurality of physiotherapy instrument hosts first, and operation instructions are directionally transmitted to the host, wherein the operation instructions comprise operation instructions to be executed by the host and other hosts. After the host receives the control command sent by the physiotherapy instrument control end, the control sub-command to be executed by the host is executed, and the control sub-commands to be executed by other hosts are distributed to other hosts at the same time. By confirming a distribution way of the data transfer host, high-frequency use of the physiotherapy instrument control end is avoided, which improves the use time and efficiency of the physiotherapy instrument control end. Moreover, any host may become the transfer host to be connected with the control end, which may avoid the consumption of the host caused by long-term use of one transfer host.

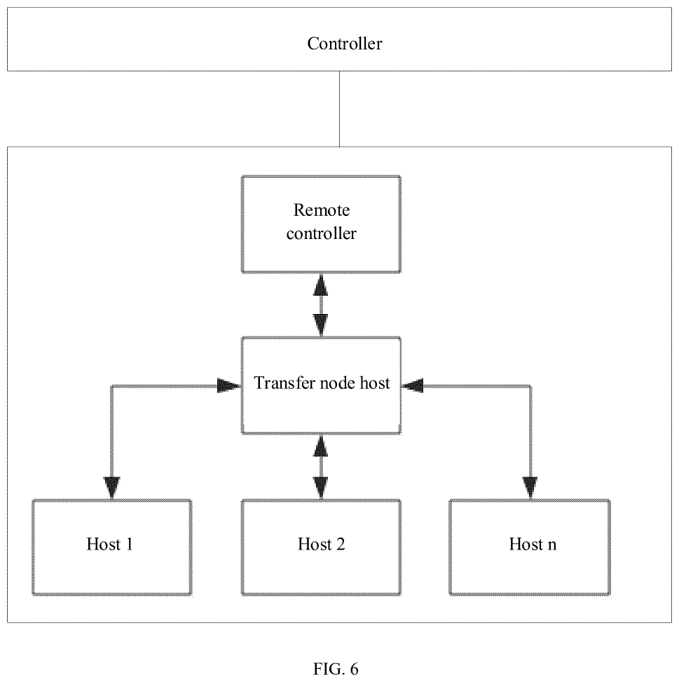

FIG. 6 shows a network structure of the wireless physiotherapy instrument control system. As shown in FIG. 6, the network structure includes a controller, a control node remote controller (the physiotherapy instrument control end), a transfer node (the data transfer host) and various common nodes (the target receiving hosts). The transfer node host is one target receiving host. The controller controls the remote controller to send the control command to the transfer node host, and the controller controls the transfer node host to send each control sub-command in the control command to the corresponding target receiving host. After the controller controls various target receiving hosts to execute the corresponding control sub-commands, execution data are fed back to the transfer node host, and the controller controls the transfer node host to send various execution data to the remote controller, so as to realize the network communication between the remote controller and various target receiving hosts, which is convenient for the remote controller to control various target receiving hosts through the transfer node host.

When the remote controller needs to control various target receiving hosts, the controller controls the remote controller of the control node to actively send the control command, and the control node only keeps connection with one transfer node at the same time. When the remote controller needs to send data to the common nodes, the remote controller cannot send the data and cannot be connected with the common nodes directly, but sends the data to the common nodes after being transferred through the transfer node. The transfer node is not limited to a target receiving host 1 or a target receiving host 2, and the target receiving host connected to the remote controller first is preferably changed to the transfer node. When the transfer node is invalid and fails to respond, the transfer node is actively switched to other available common node as the transfer node by the remote controller to continue to maintain the network communication state. When the host needs to send data, the data are also sent to the transfer node first and then reported to a remote controller control end.

One data transfer hosts is provided, which is determined according to an actual connection situation, and the data transfer host connected each time is not necessarily the same one. At least one or more target receiving hosts may be provided.

Optionally, the first determining module is specifically configured for:
acquiring transfer end information included in the control command; and
determining a first target host matched with the transfer end information in the plurality of physiotherapy instrument hosts, and taking the first target host as the data transfer host.

Optionally, the first determining module is specifically configured for:
determining the first target host matched with the transfer end information in the plurality of physiotherapy instrument hosts, and taking the first target host as the data transfer host, which includes:
acquiring host information of each physiotherapy instrument host in the plurality of physiotherapy instrument hosts, wherein the host information includes synchronization byte information and channel information;
taking host information which is the same as the transfer end information in multiple pieces of host information as first target host information; and
taking a physiotherapy instrument host corresponding to the first target host information as the first target host, and taking the first target host as the data transfer host.

Optionally, the second determining module is specifically configured for:
aiming at each control sub-command in the plurality of control sub-commands, acquiring receiving end information included in each control sub-command; and
determining a second target host matched with the receiving end information in the plurality of physiotherapy instrument hosts, and taking the second target host as the target receiving host corresponding to each control sub-command.

Optionally, the second determining module is specifically configured for:
determining the second target host matched with the receiving end information in the plurality of physiotherapy instrument hosts, and taking the second target host as the target receiving host corresponding to each control sub-command, which includes:
acquiring host information of each physiotherapy instrument host in the plurality of physiotherapy instrument hosts, wherein the host information includes synchronization byte information and channel information;
taking host information which is the same as the receiving end information in multiple pieces of host information as second target host information; and
taking a physiotherapy instrument host corresponding to the second target host information as the second target host, and taking the second target host as the target receiving host corresponding to each control sub-command.

Optionally, the system further includes an exception handling module, and the exception handling module is specifically configured for:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands included in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

Optionally, the exception handling module is specifically configured for:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

A computing device is provided in an embodiment of the present disclosure, which includes a memory, a processor, and a program stored on the memory and operated on the processor, wherein the processor executes the program to implement some or all the steps of the wireless physiotherapy instrument control method.

The computing device may be a computer, and correspondingly, the program is computer software. Various parameters and steps in the computing device according to the present disclosure above may refer to various parameters and steps in the embodiment of the wireless physiotherapy instrument control method above, which will not be repeated herein.

A computer-readable storage having an instruction stored therein is provided in an embodiment of the present disclosure, wherein the computer-readable storage medium stores an instruction, and when the instruction is operated, the steps of the wireless physiotherapy instrument control method above are executed.

The computer-readable storage medium may be a transient computer-readable storage medium or a non-transient computer-readable storage medium.

The technical solution in the embodiment of the present disclosure may be embodied in a form of a software product, the computer software product is stored in a storage medium including one or more instructions to make a computer device (which may be a personal computer, a server, a network device, or the like) execute all or some steps of the method in the embodiment of the present disclosure. The foregoing computer-readable storage medium may be the non-transient computer-readable storage medium, including: multiple media capable of storing program codes such as a USB disk, a mobile hard disk, a Read-Only Memory (ROM), a Random Access Memory (RAM), a magnetic disk or an optical disk, or the transient computer-readable storage medium.

The flow chart and the block diagram in the drawings show system architectures, functions and operations that are possible to be implemented according to the system, the method and the computer program product according to various embodiments of the present disclosure. Each block in the flow charts or the block diagrams may represent one module, one program segment, or a part of code, and the module, the program segment, or the part of code above contains one or more executable instructions for implementing specified logical functions. It should also be noted that in some alternative implementations, the functions noted in the blocks may also occur in a different order from those noted in the drawings. For example, two consecutively shown blocks may actually be executed in substantially parallel, and sometimes may be executed in a reverse order, depending on the functions involved. It should also be noted that each block in the block diagrams or the flow charts, and the combinations of the blocks in the block diagrams or the flow charts may be implemented with dedicated hardware-based systems that execute specified functions or operations, or may be implemented with the combinations of dedicated hardware and computer instructions.

Those skilled in the art know that the present disclosure may be implemented as the system, the method or the computer program product. Therefore, the present disclosure can be specifically implemented in the following form, that is, it may be complete hardware, complete software (including firmware, resident software, microcode, and the like), or a combination of hardware and software, which is generally referred to as “circuit”, “module” or “system” herein. Furthermore, in some embodiments, the present disclosure can also be implemented in a form of a computer program product in one or more computer-readable media, and the computer-readable medium contains computer-readable program codes. The computer-readable storage medium may be, but is not limited to, for example, an electrical, magnetic, optical, electromagnetic, infrared, or semiconductor system, apparatus or device, or any combination of the above.

In the descriptions of the specification, the descriptions with reference to the terms such as “one embodiment”, “some embodiments”, “example”, “specific example” or “some examples” refer to that specific features, structures, materials or characteristics described with reference to the embodiments or examples are included in at least one embodiment or example of the present disclosure. In the specification, the schematic representation of the above terms does not necessarily refer to the same embodiment or example. Moreover, the specific features, structures, materials or characteristics described may be combined in any one or more embodiments or examples in a suitable manner. In addition, in the case of having no mutual contradiction, those skilled in the art may join and combine different embodiments or examples described in the specification and the characteristics of the different embodiments or examples.

Although the embodiments of the present disclosure have been shown and described above, it can be understood that the above embodiments are exemplary and cannot be understood as limiting the present disclosure, and those of ordinary skills in the art may make changes, modifications, substitutions and variations to the above embodiments within the scope of the present disclosure.

What is claimed is:

1. A wireless physiotherapy instrument control method, comprising the following steps of:
acquiring a control command sent by a physiotherapy instrument control end;
determining a data transfer host corresponding to the control command from a plurality of physiotherapy instrument hosts;
acquiring a plurality of control sub-commands comprised in the control command;
aiming at each control sub-command in the plurality of control sub-commands, determining a target receiving host corresponding to each control sub-command from the plurality of physiotherapy instrument hosts;
receiving the control command by using the data transfer host, and transferring each control sub-command in the control command to the corresponding target receiving host; and
controlling each target receiving host to execute each corresponding control sub-command.

2. The method according to claim 1, wherein, the determining the data transfer host corresponding to the control command from the plurality of physiotherapy instrument hosts, comprises:
acquiring transfer end information comprised in the control command; and
determining a first target host matched with the transfer end information in the plurality of physiotherapy instrument hosts, and taking the first target host as the data transfer host.

3. The method according to claim 2, wherein the transfer end information comprises transfer end synchronization byte information and transfer end channel information; and
the determining the first target host matched with the transfer end information in the plurality of physiotherapy instrument hosts, and taking the first target host as the data transfer host, comprises:
acquiring host information of each physiotherapy instrument host in the plurality of physiotherapy instrument hosts, wherein the host information comprises synchronization byte information and channel information;
taking host information which is the same as the transfer end information in multiple pieces of host information as first target host information; and
taking a physiotherapy instrument host corresponding to the first target host information as the first target host, and taking the first target host as the data transfer host.

4. The method according to claim 1, wherein, the aiming at each control sub-command in the plurality of control sub-commands, determining the target receiving host corresponding to each control sub-command from the plurality of physiotherapy instrument hosts, comprises:
aiming at each control sub-command in the plurality of control sub-commands, acquiring receiving end information comprised in each control sub-command; and
determining a second target host matched with the receiving end information in the plurality of physiotherapy instrument hosts, and taking the second target host as the target receiving host corresponding to each control sub-command.

5. The method according to claim 4, wherein the receiving end information comprises receiving end synchronization byte information and receiving end channel information; and
the determining the second target host matched with the receiving end information in the plurality of physiotherapy instrument hosts, and taking the second target host as the target receiving host corresponding to each control sub-command, comprises:
acquiring host information of each physiotherapy instrument host in the plurality of physiotherapy instrument hosts, wherein the host information comprises synchronization byte information and channel information; and
taking host information which is the same as the receiving end information in multiple pieces of host information as second target host information; and
taking a physiotherapy instrument host corresponding to the second target host information as the second target host, and taking the second target host as the target receiving host corresponding to each control sub-command.

6. The method according to claim 1, further comprising the following steps of:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands comprised in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

7. The method according to claim 6, wherein the when the data transfer host is in the abnormal state, acquiring the new control command sent by the physiotherapy instrument control end, comprises:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

8. The method according to claim 2, further comprising the following steps of:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands comprised in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

9. The method according to claim 8, wherein the when the data transfer host is in the abnormal state, acquiring the new control command sent by the physiotherapy instrument control end, comprises:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

10. The method according to claim 3, further comprising the following steps of:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands comprised in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

11. The method according to claim 10, wherein the when the data transfer host is in the abnormal state, acquiring the new control command sent by the physiotherapy instrument control end, comprises:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

12. The method according to claim 4, further comprising the following steps of:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands comprised in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

13. The method according to claim 12, wherein the when the data transfer host is in the abnormal state, acquiring the new control command sent by the physiotherapy instrument control end, comprises:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

14. The method according to claim 5, further comprising the following steps of:
when the data transfer host is in an abnormal state, acquiring a new control command sent by the physiotherapy instrument control end;
determining a new data transfer host corresponding to the new control command from the plurality of physiotherapy instrument hosts;
acquiring a plurality of new control sub-commands comprised in the new control command;
aiming at each new control sub-command in the plurality of new control sub-commands, determining a new target receiving host corresponding to each new control sub-command from the plurality of physiotherapy instrument hosts;
receiving the new control command by the new data transfer host, and transferring each new control sub-command in the new control command to the corresponding new target receiving host; and
controlling each new target receiving host to execute each corresponding new control sub-command.

15. The method according to claim 14, wherein the when the data transfer host is in the abnormal state, acquiring the new control command sent by the physiotherapy instrument control end, comprises:
controlling the physiotherapy instrument control end to send an inquiry instruction to the data transfer host;
after the physiotherapy instrument control end does not receive response information fed back by the data transfer host based on the inquiry instruction, controlling the physiotherapy instrument control end to poll and send the inquiry instruction according to a preset period;
recording a number of sending times of the inquiry instruction polled and sent, and after the physiotherapy instrument control end receives the response information fed back by the data transfer host based on the inquiry instruction, resetting the number of sending times of the inquiry instruction; and
when the number of sending times reaches a preset threshold, determining that the data transfer host is in the abnormal state, and acquiring the new control command sent by the physiotherapy instrument control end.

16. A computing device, comprising a memory, a processor, and a program stored on the memory and operated on the processor, wherein the processor executes the program to implement the steps of the wireless physiotherapy instrument control method according to claim 1.

17. A computer-readable storage medium having an instruction stored therein, which when running on a terminal device, causes the terminal device to perform the steps of the wireless physiotherapy instrument control method according to claim 1.
